(19) United States
(12) Patent Application Publication
(10) Pub. No.: US 2009/0043315 A1
(43) Pub. Date: Feb. 12, 2009

(54) Endobag for endoscopic surgery

(76) Inventor: Hwa-sook Moon, Busan (KR)

Correspondence Address: John W. Renner, Esq.;Renner, Otto, Boiselle & Sklar LLp, 19th Floor, 1621 Euclide Avenue, Cleveland, OH, 44115, US

(21) Appl. No.: 12/074,278

(22) Filed: Mar. 3, 2008

(30) Foreign Application Priority Data
Aug. 6, 2007 (KR) 10-2007-0078737

(51) Int. Cl. A61B 19/00

(52) U.S. Cl. 606/114

(57) ABSTRACT

Disclosed is an endobag for endoscopic surgery. The endobag comprises a vinyl bag, which includes harmless transparent vinyl material and is prepared such that an upper end of the vinyl bag forms an inlet, and a pair of flaps that protrude from the inlet of the folded vinyl bag such that the vinyl bag inserted into an abdomen through a trocar is easily unfolded. The flaps have a color for indicating the inlet of the vinyl bag and protrude from front and rear sides of the inlet of the vinyl bag while being offset from each other.

### TECHNICAL FIELD

The present invention relates to an endobag that is inserted into the human body during abdominal surgery using an endoscope to extract tumor or internal organs, which are removed during the abdominal surgery, out of an abdomen by receiving the tumor or internal organs in the vinyl bag. More particularly, the present invention relates to an endobag that comprises a vinyl bag made from harmless transparent vinyl material and a pair of flaps protruding from front and rear sides of an inlet of the vinyl bag such that they are offset from each other, so that the inlet of the vinyl bag can be opened more quickly and easily in an abdomen, and extract materials such as tumor or internal organs received in the vinyl bag can be more easily taken out of the abdomen by using the flaps in a state in which the inlet of the vinyl bag is withdrawn out of the abdomen, thereby improving the convenience of an operator performing abdominal surgery and preventing the abdomen of a patient from being damaged.

### BACKGROUND ART

In general, an endoscope refers to medical instruments for allowing an operator to directly observe the inner surfaces of hollow organs such as the stomach or esophagus, or the inner parts of a human body such as the thorax or an abdomen. An endoscope is variously classified as a bronchoscope, a laryngoscope, an esophagoscope, a stomach camera, a hysteroscope, a urethroscope, an arthroscope, a cystoscope, a rectoscope, a peritoneoscope or a cardioscope according to purposes thereof.

When the endoscope is used for abdominal surgery as described above, an operator can easily extract internal organs or tumor while observing the internal state of an abdomen. Accordingly, the operator can conveniently diagnose and treat parts requiring a surgical operation. In addition, abdominal walls and abdominal organs are minimally damaged during a surgical operation, so that complications, recovery time of a patient and pain during the recovery of the patient can all be reduced.

During the abdominal surgery using an endoscope as described above, when an operator performs an operation of removing tumors or suturing internal organs and the like, the operator inserts various medical instruments into an abdomen, together with the endoscope and surgical instruments, to take pieces of tumors or internal organs, which are removed during the abdominal surgery, out of the abdomen by receiving the pieces of tumors or internal organs in a vinyl bag using a medical instrument referred to as a lap-bag.

As shown in FIG. 1, the conventional lap-bag used for the abdominal surgery using the endoscope as described above comprises an insert tube 2, an auxiliary tube 5, an insert rod 6 and a wire spring 4. The auxiliary tube 5 and the insert rod 6 are sequentially inserted into the insert tube 2 having a rod shape and have knobs 5a and 6a at one end thereof, respectively. The wire spring 4 is integrally connected with the front end of the insert rod 6.

The wire spring 4 is incorporated with a vinyl bag 3 comprising transparent material harmless to the human body. After the vinyl bag 3 is initially inserted into the insert tube 2 in a state in which the vinyl bag 3 is folded, if the insert rod 6 is inserted into the insert tube 2 together with the auxiliary tube 5, the vinyl bag 3 is taken out of the insert tube 2 together with the wire spring 4 and then an inlet of the vinyl bag 3 is opened by the wire spring 4 as shown in FIG. 1.

Further, the wire spring 4 is inserted into the space defined at the upper portion of the vinyl bag 3 by a sealing section 3a. As the insert rod 6 is completely withdrawn from the auxiliary tube 5, the wire spring 4 is come out of the insert tube 2 together with the insert rod 6 via the vinyl bag 3 and the auxiliary tube 5 through a ∥-shaped guide groove (not shown) formed at the front end of the auxiliary tube 5.

A pulling string 7 is inserted into the space, which is defined at the upper portion of the vinyl bag 3 by the sealing section 3a, together with the wire spring 4. The pulling string 7 forms a ring-shaped knot at the front end of the auxiliary tube 5, and then extends out of the insert tube 2 along the space between the auxiliary tube 5 and the insert rod 6 by passing through the front end surface of the auxiliary tube 5, so that the sealing of the vinyl bag 3 can be finally achieved by means of the pulling string 7. The pulling string 7 is provided with a pulling ring 8 at the free end thereof.

Before describing a surgical operation of extracting pieces of tumors or internal organs, which are removed during the abdominal surgery, out of the abdomen by receiving the pieces of tumors or internal organs by means of the conventional lap-bag 1 having the construction as described above, a 10 mm trocar will be described as an endoscopic surgery tool, which forms pneumoperitoneum by putting gas (e.g. CO2 gas) into the abdomen during endoscopic surgery so as to create the space and visibility for the endoscopic surgery, and allows an endoscope, a surgical instrument and a medical instrument, such as an endoloop or the lap-bag 1, to be inserted into the abdomen.

As shown in FIG. 2, the trocar 10 comprises an obturator 11 having a cylindrical shape, a cannula 12, a gas injector 13 provided with a valve knob 14, and a packing cover 15 made from rubber. The gas injector 13 is connected to one side of the obturator 11. The cannula 12 is used for inserting surgical instruments into an abdomen and is installed through the obturator 11. The packing cover 15 has an insertion hole 15a, into which the surgical instruments are inserted, at the central portion thereof. The packing cover 15 is inserted into the upper portion of the obturator 11 through a connection member 16.

The obturator 11 blocks the back flow of CO₂ gas injected into the abdomen through the cannula 12, and simultaneously, allows various surgical instruments to be inserted through the cannula 12. The packing cover 15 allows an endoscope, which has a diameter of about 10 mm, surgical instruments, an endoloop or the lap-bag 1 to be inserted into the abdomen while maintaining the air-tightness.

However, since the endoscope, the surgical instruments, the endoloop or the lap-bag 1 may have a diameter of about 5 mm, an auxiliary cannula 17 is inserted into the trocar 10 in order to allow such tools to be inserted through the trocar 10 while maintaining the air-tightness. A packing cover 19 comprising rubber is installed at the upper portion of the auxiliary cannula 17 similarly to the 10 mm trocar 10. The packing cover 19 has an insertion hole 19a at the central portion thereof such that instruments having a diameter of about 5 mm can be inserted into the packing cover 19 while maintaining the air-tightness. Further, a flange-shaped plate 18 for covering the packing cover 15 of the 10 mm trocar 10 is installed below the packing cover 19.

Hereinafter, an operation process for taking pieces of tumor or internal organs removed during abdominal surgery out of an abdomen by using the conventional lap-bag 1 and trocar 10 as described above will be described in detail with reference to FIG. 3.

First, as shown in FIG. 3a, the front end of the insert tube 2 of the lap-bag 1 is inserted into an abdomen 9 through the trocar 10 inserted into the abdomen 9. Then, as the insert rod 6 extending backward from the insert tube 2 is pushed into the insert tube 2 together with the auxiliary tube 5, the vinyl bag 3 is ejected through the front end of the insert tube 2, and simultaneously, an inlet of the vinyl bag 3 is opened by the elastic force of the wire spring 4 as shown in FIG. 3b.

In such a state, surgical instruments, such as scissors or nippers, are inserted through another trocar (not shown), pieces of tumors or internal organs removed during the abdominal surgery are properly cut using the surgical instruments, and are received in the vinyl bag 3. Then, as the insert rod 6 is completely ejected from the auxiliary tube 5 as shown in FIG. 3c, the wire spring 4 inserted into the vinyl bag 3 is ejected together with the insert rod 6, and simultaneously, the inlet of the vinyl bag 3 is narrowed by a certain degree.

In such a state, as shown in FIG. 3d, as the pulling ring 8 of the pulling string 7 that extends outward through the auxiliary tube 5 is pulled, the inlet of the vinyl bag 3 is completely narrowed. Since the operation using the lap-bag 1 corresponds to the final step of the abdominal surgery, the pieces of tumors or internal organs removed during the abdominal surgery can be taken out of the abdomen 9 together with the trocar 10 with receiving tumors or internal organs in the vinyl bag 3.

However, the conventional lap-bag 1 has a complicated structure in which the vinyl bag 3 is incorporated with the wire spring 4 connected to the front end of the insert rod 6 after the auxiliary tube 5 and the insert rod 6 are sequentially inserted into the insert tube 2 serving as a main tube, causing an increase in the cost of the lap-bag 1. Thus, operating charges for a simple operation such as appendectomy may increase. In addition, since the operation process using the lap-bag 1 is relatively complicated, an operator must carefully perform the surgical operation.

In addition, although the space for the surgical operation is created by injecting gas into the abdomen 9 through the 10 mm trocar 10, the inner part of the abdomen 9 or the internal organs may be damaged by the wire spring 4 comprising metal or a rigid plastic rod and the like in the process of inserting the vinyl bag 3 and opening the inlet of the vinyl bag 3 provided in the lap-bag 1. Since the lap-bag 1 used for the abdominal surgery cannot be reused due to the characteristics of medical instruments, the lap-bag 1 must be discarded. Thus, medical resources are unnecessarily wasted and environmental contamination is caused by medical waste. In addition, when the conventional lap-bag 1 is taken out of the abdomen 9 with receiving pieces of internal organs or tumors, since the inlet of the vinyl bag 3 must be fully ejected simultaneously, a vinyl bag having a relatively large size may not be effectively used.

In particular, when a peritoneoscope operation is performed, three or four trocars 10 are inserted into the abdomen 9 such that an endoscope or surgical instruments can be inserted into the abdomen 9. However, since an endoscope or other surgical instruments cannot be inserted through the trocar 10 into which the lap-bag 1 is inserted, the number of trocars 10, through which an endoscope or surgical instruments can be inserted, is relatively reduced. Thus, it may be difficult to properly cut tumor or pieces of internal organs and receive the cut tumors or internal organs in the vinyl bag 3 of the lap-bag 1.

In order to compensate for the problems, an additional 10 mm trocar for insertion of the lap-bag 1 may also be inserted into the abdomen 9 of a patient. However, this may cause unnecessary surgical damage of the abdomen 9, increase of the total operation time required for the abdominal surgery and exert a bad influence on the quick recovery of a patient.

In order to solve the problems of the conventional lap-bag 1, a vinyl bag 21 comprising harmless transparent vinyl material is provided. The vinyl bag 21 is prepared such that an upper end of the vinyl bag 21 forms an inlet 22. Further, the vinyl bag 21 has an inlet indication band 23 around the inlet 22 such that the folded vinyl bag 21 inserted into the abdomen 9 through the trocar 10 can be easily unfolded. An endobag 20 for endoscopic surgery provided with a folding section 24, by which the inlet 21 of the vinyl bag 21 is folded one time, is disclosed in Korean Patent Registration No. 10-729444 (registration date: Jun. 11, 2007), which was filed with Korean Intellectual Office on Dec. 16, 2005 with Patent Application No. 2005-124523 by present applicant.

According to the previously filed endobag 20 for endoscopic surgery, only the inlet indication band 23 is formed around the inlet 22 of the vinyl bag 21, so that the number of core parts necessary for removing extract materials generated during the abdominal surgery can be reduced, as compared with the conventional lap-bag 1. Thus, the endobag 20 can be manufactured at a low cost as compared with the conventional lap-bag 1. In addition, an operation process for removing the extract materials can be simplified by using the inlet indication band 23 and the folding section 24. As a result, the cost required for the abdominal surgery can be saved and the convenience of an operator can be significantly improved.

Further, in a state in which the space for the surgical operation is created by injecting gas in the abdomen 9 through the trocar 10, only the endobag 20 comprising smooth vinyl material is simply inserted into the abdomen 9, so that the inner part of the abdomen 9 and the internal organs can be prevented from being damaged in the process of taking the extract materials generated during the abdominal surgery out of the abdomen 9, as compared with the conventional lap-bag 1 through which a metal spring or a rigid plastic rod is inserted into the abdomen 9. In addition, since only the endobag 20 is discarded after the abdominal surgery, unnecessary waste of resources and environmental contamination caused by medical waste can be minimized.

In particular, since the previously filed endobag 20 is pushed into the abdomen 9 through a trocar by using various surgical instruments, the number of the conventional trocars 10 is not relatively reduced due to insertion of the endobag 20. Therefore, an operation of receiving tumors or internal organs in the vinyl bag 21 can be performed more easily.

Accordingly, since it is not necessary to insert an additional trocar into the abdomen 9 such that the vinyl bag 3 used for receiving extract materials can be inserted through the insert tube 2, the abdomen 9 can be prevented from being unnecessarily damaged during the abdominal surgery. In addition, the total operation time required for the abdominal surgery can be minimized and quick recovery of a patient can be achieved.

Further, various sizes of endobags 20 can be selectively used according to the amount of tumors or internal organs which must be removed. Furthermore, the endobag 20 having a relatively large size can be easily inserted into the abdomen 9 through the trocar 10 and the inlet 22 of the endobag 20 can be taken out of the abdomen 9 more easily and safely. Moreover, in a state in which the endobag 20 is inserted into the abdomen 9, removed tumors or internal organs are received in the vinyl bag 21 without cutting them, so that other internal organs in the abdomen 9 can be prevented from being contaminated by the tumors or internal organs removed during the abdominal surgery. In addition, in a state in which the removed tumors or internal organs are received in the endobag 20, other surgical instruments can be inserted into the abdomen 9 through the trocar 10 to perform other types of surgical operations.

However, since the endobag 20 has some disadvantages in its application process in spite of many advantages as described above. First, in a state in which the endobag 20 is inserted into the abdomen 9, the position of the inlet 22 of the vinyl bag 21 can be easily found by the inlet indication band 23. However, if the thin vinyl endobag 20 is covered with bloods or body fluids generated during the abdominal surgery, the inlet 22 of the vinyl bag 21 may cohere with the bloods or body fluids, so it is difficult to open the inlet 22 by using surgical instruments.

If the inlet 22 of the vinyl bag 21 is not easily opened in the abdomen 9 as described above, time required for endoscope surgery using the endobag 20 may be increased and an operator may feel inconvenienced. In addition, in the process of opening the inlet 22 of the vinyl bag 21 covered with bloods or body fluids by using surgical instruments, the surgical instruments may unnecessarily touch internal organs in the abdomen 9 of a patient to cause complications. Thus, quick recovery of a patient may be delayed.

Second, when the inlet 22 of the vinyl bag 21 receiving extract materials such as tumors or internal organs is withdrawn out of the abdomen 9 together with the trocar 10 after the abdominal surgery using an endoscope, opening the inlet 22 of the vinyl bag 21 in the outside of the abdomen 9 may also cause inconvenience to an operator because the inlet 22 of the vinyl bag 21 coheres with bloods or body fluids. Thus, the operation time using an endoscope may be further increased and an operator may feel inconvenienced.

Disclosure

Technical Problem

The present invention has been made to solve the above problems occurring in the prior art, and an object of the present invention is to provide an endobag for endoscopic surgery which is provided with a vinyl bag comprising harmless transparent vinyl material, and allows an inlet of the vinyl bag covered with bloods or body fluids to be quickly and easily opened using flaps.

Technical Solution

In order to accomplish the above object, according to one aspect of the present invention, there is provided an endobag for endoscopic surgery, which takes tumors or internal organs removed during abdominal surgery out of an abdomen through a trocar used for abdominal surgery by receiving the tumors or internal organs in a vinyl bag, the endobag comprising: the vinyl bag including harmless transparent vinyl material and being prepared such that an upper end of the vinyl bag forms an inlet; and a pair of flaps protruding from the inlet of the folded vinyl bag such that the vinyl bag inserted into the abdomen through the trocar is easily unfolded, wherein the flaps have a color for indicating the inlet of the vinyl bag and protrude from front and rear sides of the inlet of the vinyl bag while being offset from each other.

According to the endobag, both sides of an upper end of the vinyl bag are cut in a downward direction to form cutting parts such that the inlet is easily opened using the flaps.

In a state in the flaps of an endobag, which is inserted into an abdomen through a 10 mm trocar, are clamped by using surgical instruments, the inlet of the endobag can be quickly and easily opened. Further, after the inlet of the endobag is withdrawn out of the abdomen together with the trocar while one edge of the endobag is being clamped, the inlet of the endobag withdrawn out of the abdomen can be easily opened using the flaps. In such a state, tumors or internal organs received in the endobag are cut using the surgical instruments such that the tumors or internal organs are gradually taken out of the endobag, so that the remaining pieces of tumors or internal organs can be taken out of the abdomen together with the endobag.

Advantageous Effects

As described above, an endobag according to the present invention has advantages and additional effects of the previously filed endobag. Further, in a state in which the endobag is inserted into an abdomen, the position of an inlet of a vinyl bag can be easily indicated by flaps. In addition, even if the inlet of the thin vinyl endobag is covered with bloods or body fluids in an abdomen, the inlet of the vinyl bag can be opened more quickly and easily by clamping the flaps using various surgical instruments.

Further, the inlet of the vinyl bag can be opened more quickly and easily in the abdomen, so that unnecessary contact between internal organs in the abdomen of a patient and surgical instruments can be minimized in the process of opening the inlet of the thin vinyl endobag covered with bloods or body fluids by using the surgical instruments. Thus, the abdomen can be prevented from being additionally damaged during the surgical operation, so that quick recovery of a patient can be achieved.

In addition, when performing an operation of removing extract materials, such as tumors or internal organs, received in the vinyl bag by taking the inlet of the vinyl bag out of the abdomen together with the trocar after the abdominal surgery using an endoscope, the inlet of the thin vinyl bag of the endobag, which is covered with bloods or body fluids, can be opened quickly and easily by using the flaps. Thus, the operation time using the endoscope can be shortened and the convenience of an operator can be significantly improved. Moreover, cutting parts are formed at the upper ends of both sides of the vinyl bag together with the flaps, so that the inlet of the thin vinyl bag can be more easily opened using the flaps.

### DESCRIPTION OF THE DRAWINGS

The above and other advantages of the present invention will become readily apparent with reference to the following detailed description when considered in conjunction with the accompanying drawings wherein:

FIG. 1 is a perspective view illustrating a conventional lap-bag used for endoscopic surgery;

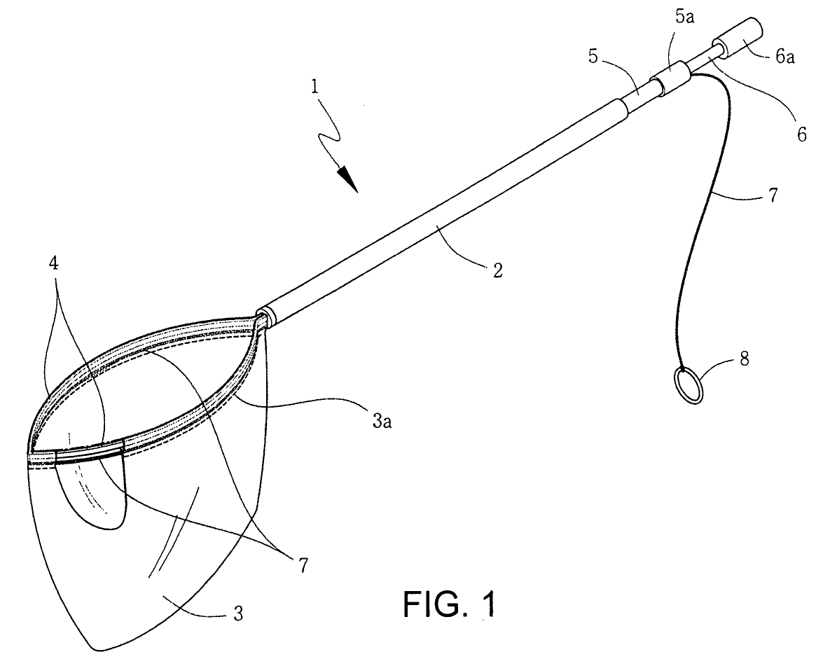

FIG. 2 is a perspective view illustrating a trocar used for endoscopic surgery;

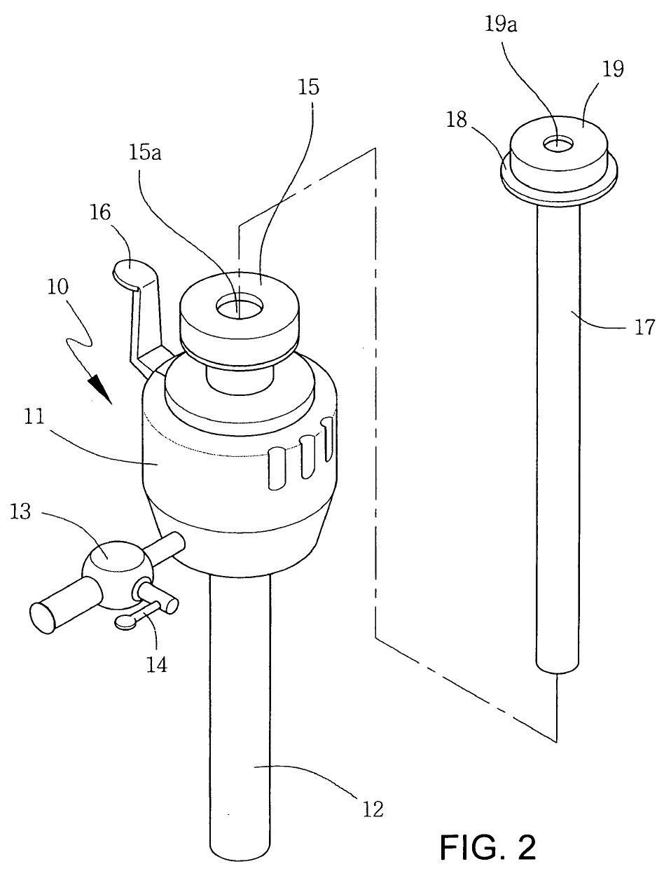

FIG. 3 are views illustrating a process of performing abdominal surgery using a conventional lap-bag;

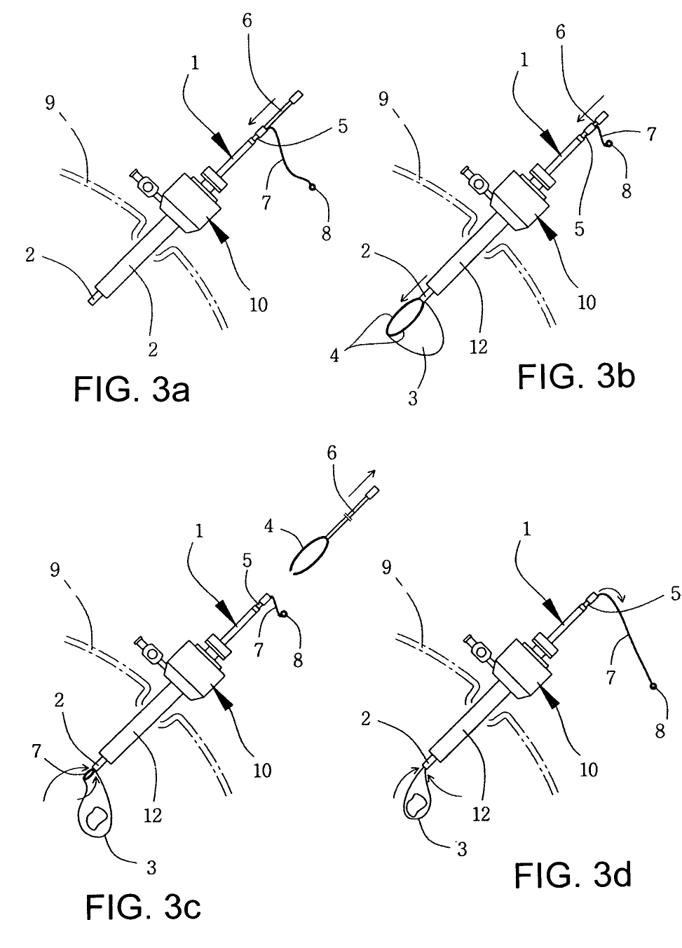

FIGS. 4a and 4b are perspective views illustrating an endobag for endoscopic surgery according to the prior art;

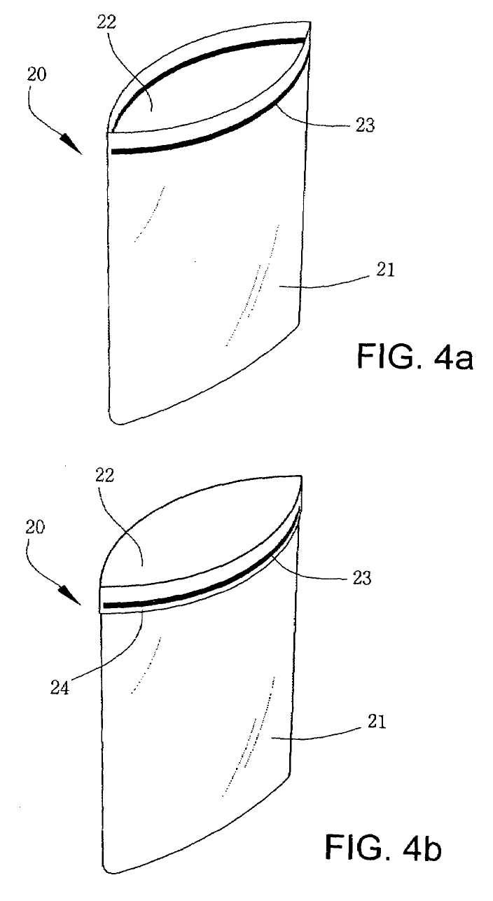

FIGS. 5a and 5b are perspective views illustrating an endobag for endoscopic surgery according to the present invention;

FIGS. 6a and 6b are views illustrating a process of inserting an endobag according to the present invention into an abdomen; and

FIGS. 7a to 7d are views illustrating a process of extracting material generated during abdominal surgery out of an abdomen by using an endobag according to the present invention.

### BEST MODE

Hereinafter, a preferred embodiment of an endobag for endoscopic surgery according to the present invention will be described in detail with reference to the accompanying drawings.

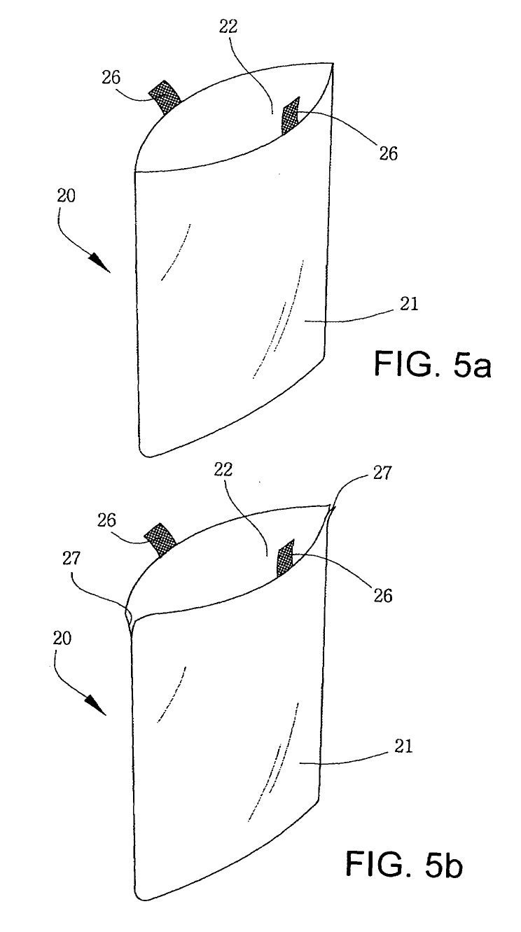

The endobag 20 for endoscopic surgery according to the present invention comprises a rectangular-shaped vinyl bag 21 including harmless transparent vinyl material, and a pair of flaps 26 as shown in FIGS. 5a and 5b. The vinyl bag 21 is prepared such that an upper end of the vinyl bag 21 forms an inlet 22. The flaps 26 are installed at the inlet 22 to allow the folded vinyl bag 21, which is inserted into an abdomen 9 through a trocar 10, to be easily unfolded.

The flaps 26 have a color for indicating the inlet 22 of the folded vinyl bag 21, similarly to the inlet indication band 23 of the previously filed endobag 20, and protrude from front and rear sides of the inlet 22 while being offset from each other and spaced apart from each other such that they do not make contact with each other when viewed from FIGS. 5a and 5b. Further, both sides of the upper end of the vinyl bag 21 are cut in the downward direction to form cutting parts 27 such that the inlet 22 can be easily opened using the flaps 26.

Similarly to the previously filed endobag 20, the endobag 20 of the present invention uses vinyl material the same as or similar to that of the vinyl bag 3 used for the conventional lap-bag 1. For example, the endobag 20 of the present invention representatively uses polyethylene resin extensively used as vinyl for food storage, latex used for gloves for surgical operation, or special vinyl used for artificial anus or artificial valve in internal operation. In addition, it is possible to use general harmless transparent vinyl by sterilizing the transparent vinyl. It is preferred to use thin and tough material if possible.

Further, preferably, the vinyl bag 21 of the endobag 20 according to the present invention uses vinyl having a relatively high transparence such that the vinyl bag 21 can easily receive tumors or internal organs removed during surgical operation. Further, it is also possible to use semi-transparent vinyl if an operator can check extract materials received in the vinyl bag 21 through an endoscope with naked eyes.

The flaps 26, which are main elements of the present invention, can also be integrally formed with the vinyl bag 21 when the vinyl bag 21 is manufactured. Further, the flaps 26 can also be formed by thermally fusing or bonding a short vinyl band having a tape shape to the surface of the inlet 22 of the vinyl bag 21. However, it is preferred not to use such a bonding scheme of a vinyl band using adhesive in consideration of the condition in which the endobag 20 is inserted into the abdomen 9 of a patient.

Preferably, the flaps 26 have a blue, green or black-based color such that the inlet 22 of the vinyl bag 21 can be more quickly recognized through the flaps 26. Preferably, pigment coated on the surface of the flaps 26 uses harmless natural pigment. More preferably, both surfaces of each flap 26 have a color in terms of the convenience of operation.

In addition, the flaps 26 are offset from each other such that the inlet 22 of the vinyl bag 21 can be easily opened using the flaps 26 as described above. An interval between the flaps 26 is changeable according to the size of the vinyl bag 21. Preferably, the interval is about 2 cm to 5 cm if possible. This is because the flaps 26 may overlap with each other in an interval less than 2 cm and precise operation may not be performed due to an increased gap between surgical instruments during endoscopic surgery in an interval exceeding 5 cm.

Further, the cutting parts 27, which facilitate opening of the inlet 22 through the flaps 26, are formed by cutting left and right upper end portions of the sealed rim of the vinyl bag 21, except for the inlet 22, in the downward direction by a predetermined length. Here, the cutting parts 27 may have a length which is changeable according to the size of the vinyl bag 21. Preferably, the cutting parts 27 have a length within the range of minimum 0.5 cm to maximum 3 cm. This is because open of the inlet 22 through the flaps 26 can be facilitated, and simultaneously, the storage space for extract materials can be sufficiently created in the vinyl bag 21.

The endobag 20 of the present invention as described above can be manufactured in various sizes according to the type of abdominal surgery. Preferably, the endobag 20 having a size of about 10 cm(breadth)×10 cm(length) is used for a simple operation such as appendectomy. In such a case, it is preferable that the flap has a size of about 1 cm to 1.5 cm(width)×1 cm to 1.5 cm(height). Further, the endobag 20 having a size of about 30 cm(breadth)×20 cm(length) is used for complicated abdominal surgery. In such a case, it is preferable that the flap has a size of about 2 cm to 3 cm(width)×2 cm to 3 cm(height). If the endobag 20 has an excessively large size, it is difficult to insert the endobag 20 into the abdomen 9 through the trocar 10 and to take the endobag 20 out of the abdomen 9 after the abdominal surgery.

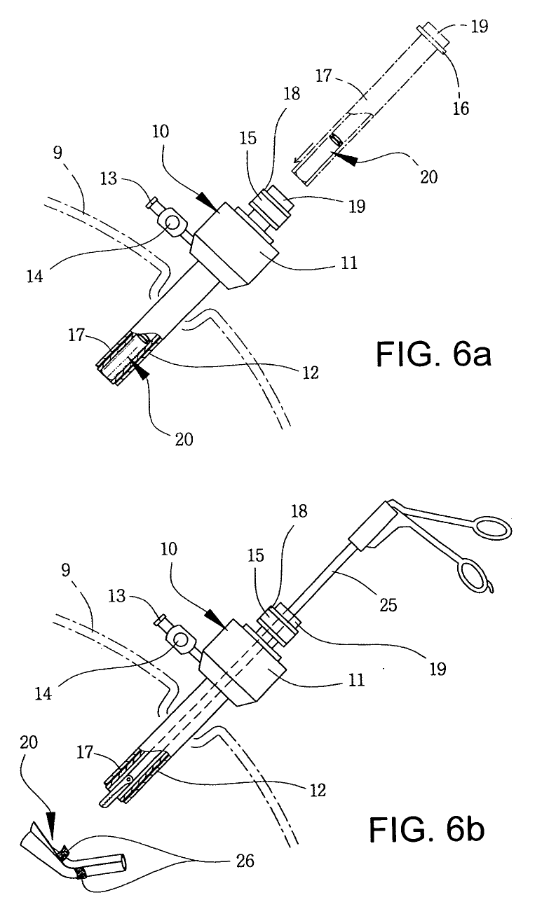

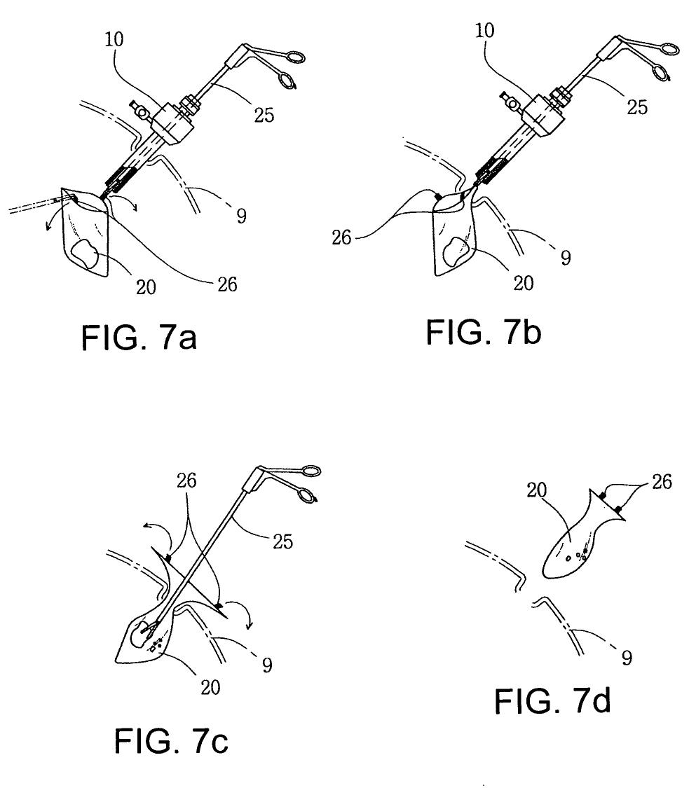

Hereinafter, an operation of the endobag 20 according to the present invention will be described in detail with reference to FIGS. 6 and 7.

First, in a state in which the endobag 20 according to the present invention is folded or rolled at a predetermined size and then is inserted into the abdomen 9 through an auxiliary cannula 17 for the 10 mm trocar 10 as shown in FIG. 6a, the auxiliary cannula 17 is primarily inserted along a cannula 12 of the trocar 10 through a packing cover 15 of the trocar 10 and then surgical instruments 25 including scissors or nippers are pushed through the auxiliary cannula 17 as shown in FIG. 6b, so that the endobag 20 in the auxiliary cannula 17 is simply inserted into the abdomen 9 in the insertion process of the surgical instruments 25.

After simply inserting the endobag 20 of the present invention into the abdomen 9 through the trocar 10, the auxiliary cannula 17 and the surgical instruments 25 as described above, the endobag 20 inserted into the abdomen 9 is unfolded using the surgical instruments 25, which are inserted through the corresponding trocar 10, and an endoscope and surgical instruments inserted in advance through another trocar (not shown), and then the flaps 26 formed at the inlet 22 of the vinyl bag 21 are pulled using the surgical instruments as shown in FIGS. 6b and 7a, so that the inlet 22 of the vinyl bag 21 can be opened more quickly and easily.

Accordingly, even if the endobag 20 inserted into the abdomen 9 is immersed in bloods or body fluids and the inlet 22 of the vinyl bag 21 is covered and coheres with the bloods or body fluids, the inlet 22 of the vinyl bag 21 can be quickly and easily opened using the surgical instruments 25, so that time required for the endoscopic surgery can be maximally shortened, thereby improving the convenience of an operation during the endoscopic surgery and minimizing unnecessary contact between internal organs in the abdomen of a patient and the surgical instruments 25.

In a state in which the inlet 22 of the vinyl bag 21 constituting the endobag 20 is opened as described above, tumors and internal organs removed during the abdominal surgery are received in the vinyl bag 21, and then the surgical instruments 25 are withdrawn along a hole formed in the abdomen 9 together with the trocar 10 while one edge of the inlet 22 of the endobag 20 is being clamped by the surgical instruments 25 inserted into the abdomen 9 through the trocar 10 as shown in FIGS. 7a and 7b, so that one edge of the inlet 22 of the endobag 20 can be easily withdrawn out of the abdomen 9 together with the 10 mm trocar 10.

In such a state, as shown in FIGS. 7c and 7d, after primarily taking the inlet 22 of the endobag 20 out of the abdomen 9 by using one edge of the endobag 20 withdrawn out of the abdomen 9, if it is necessary for the operation, the tumors or internal organs received in the endobag 20 are cut using the surgical instruments 25 such that the tumors or internal organs are gradually taken out of the endobag 20, or the contaminated body fluids or bloods received in the endobag 20 are removed using an injector (not shown), so that the remaining pieces of tumors or internal organs can be taken out of the abdomen 9 together with the endobag 20.

When such an operation is performed, the endobag 20 taken out of the abdomen 9, which has the inlet 22 covered with the bloods or body fluids, can be quickly and easily unfolded using the flaps 26. Thus, it is possible to easily and efficiently perform an operation of cutting the tumors or internal organs received in the endobag 20 by using the surgical instruments 25 and gradually taking the tumors or internal organs out of the endobag 20, or removing the contaminated body fluids or bloods received in the endobag 20 by using an injector.

Accordingly, when the endobag 20 according to the present invention is used, the operation time using an endoscope can be further shortened while maintaining all advantages of the previously filed endobag 20, and the convenience of an operator can be significantly improved. Further, the cutting parts 27 are formed at the upper end portions of both sides of the vinyl bag 21 together with the flaps 26, so that an operation of opening the inlet 22 of the vinyl bag 21 by using the flaps 26 can be performed more easily.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

1. An endobag for endoscopic surgery, which takes tumors or internal organs removed during abdominal surgery out of an abdomen through a trocar used for abdominal surgery with receiving the tumors or internal organs in a vinyl bag, the endobag comprising:
the vinyl bag comprising harmless transparent vinyl material and being prepared such that an upper end of the vinyl bag forms an inlet; and
a pair of flaps protruding from the inlet of the folded vinyl bag such that the vinyl bag inserted into the abdomen through the trocar is easily unfolded,
wherein the flaps have a color for indicating the inlet of the vinyl bag and protrude from front and rear sides of the inlet of the vinyl bag while being offset from each other.

2. The endobag as claimed in claim 1, wherein both sides of an upper end of the vinyl bag are cut in a downward direction to form cutting parts such that the inlet is easily opened using the flaps.
